(19) United States
(12) Patent Application Publication
(10) Pub. No.: US 2008/0039481 A1
(43) Pub. Date: Feb. 14, 2008

(54) Seed Dressing for Soya Bean

(75) Inventors: Konrad Kemper, Willebadessen (DE); Isolde Hauser-Hahn, Leverkusen (DE); Paul Reinecke, Leverkusen (DE)

Correspondence Address: STERNE, KESSLER, GOLDSTEIN & FOX P.L.L.C., 1100 NEW YORK AVENUE, N.W., WASHINGTON, DC, 20005, US

(73) Assignee: BAYER CROPSCIENCE AG, Monheim (DE)

(21) Appl. No.: 10/598,336

(22) PCT Filed: Jun. 7, 2005
(86) PCT No.: PCT/EP05/06085

§ 371 Date: Sep. 25, 2007

(30) Foreign Application Priority Data
Jun. 18, 2004 (EP) 04014307.5

(51) Int. Cl. A01N 43/653; A01H 5/10; A01N 43/40; A01N 43/76; A01P 3/00; A01N 55/10; A01N 43/50; A01N 43/54

(52) U.S. Cl. 514/266.23; 504/100; 514/256; 514/357; 514/374; 514/383; 514/399; 514/63; 800/312

(57) ABSTRACT

The use of one or more DMI fungicides from the group consisting of: a) triazoles: a.1. azaconazole, a.2. bitertanol, a.3. bromuconazole, a.4. cyproconazole, a.5. difenoconazole, a.6. diniconazole, a.7. epoxiconazole, a.8. fenbuconazole, a.9. fluquinconazole, a.10. flusilazole, a.11. flutriafol, a.12. hexaconazole, a.13. imibenconazole, a.14. ipconazole, a.15. metconazole, a.16. myclobutanil, a.17. paclobutrazol, a.18. penconazole, a.19. propiconazole, a.20. prothioconazole, a.21. simeconazole, a.22. tebuconazole, a.23. tetraconazole, a.24. triadimenol, a.25. triticonazole; b) pyrimidines: b.1. fenarimol, b.2. nuarimol; c) pyridiness: c.1. pyrifenox; and d) imidazoles: d.1. imazalil, d.2. oxpoconazole fumarate, d.3. peforazoate, d.4. prochloraz, d.5. triflumizole, as seed dressing for soya beans against soya bean rust.

The invention relates to the use of fungicides acting as sterol biosynthesis inhibitors as seed dressings, to a method for controlling phytopathogenic fungi by treating seed with these fungicides, and also to seed treated with such fungicides.

Inhibitors of the sterol biosynthesis in fungi which block the demethylation in position 14 in the ergosteroid biosynthesis (demethylation inhibitors, DMI) have attained great practical importance as fungicides (see, for example, C. D. S. Tomlin (Publ.), The Pesticide Manual, 13th edition, The British Crop Protection Council, Farnham 2003).

Some of these fungicides are already known as seed dressing for seed of, inter alia, soya beans (see, for example, WO-A 03/09 90 10, Fitopatologia Brasileira (1998) 23(2) 127-131). Also, in a few cases, the action of such a seed dressing against soya bean rust has been described (Fitopatologia Brasileira (1984) 9(1), 13 and 119).

US 2003/0060371 proposes the dressing of soya bean seed with, inter alia, DMI fungicides, to improve yield and strength of the plants. There is no indication of any action against soya bean rust.

However, since the ecological and economic requirements imposed on modern-day fungicides are continually increasing, with regard, for example, to the activity spectrum, toxicity, selectivity, application rate, formation of residues, and favourable preparability, and since, furthermore, there may be problems, for example, with resistances, a constant task is to develop new fungicides which in some areas at least have advantages over their known counterparts.

Surprisingly, it has now been found that fungicides from the group of the DMI, used as seed dressing for soya bean seed, are particularly effective against soya bean rust (Phakopsora, in particular Phakopsora pachyrhiziand Phakopsora meibomiae).

Accordingly, the invention provides the use of one or more DMI fungicides from the group consisting of:
a) triazoles:
   a.1. azaconazole, a.2. bitertanol, a.3. bromuconazole, a.4. cyproconazole, a.5. difenoconazole, a.6. diniconazole, a.7. epoxiconazole, a.8. fenbuconazole, a.9. fluquinconazole, a.10. flusilazole, a.11. flutriafol, a.12. hexaconazole, a.13. imibenconazole, a.14. ipconazole, a.15. metconazole, a.16. myclobutanil, a.17. paclobutrazol, a.18. penconazole, a.19. propiconazole, a.20. prothioconazole, a.21. simeconazole, a.22. tebuconazole, a.23. tetraconazole, a.24. triadimenol, a.25. triticonazole;
b) pyrimidines:
   b.1. fenarimol, b.2. nuarimol;
c) pyridines:
   c.1. pyrifenox;
      and
d) imidazoles:
   d.1. imazalil, d.2. oxpoconazole fumarate, d.3. peforazoate, d.4. prochloraz, d.5. triflumizole,
      as seed dressing for soya beans against soya bean rust.

The invention furthermore provides a method for protecting soya bean plants against soya bean rust, which method comprises treating the seed of the plants with one or more DMI fungicides from the group a-d.

Some of the DMI fungicides mentioned have hitherto not been used for dressing soya bean seed.

Accordingly, the invention also provides the use of one or more DMI fungicides from the groups a-d for dressing the seed of soya beans, a method for protecting soya bean plants against phytopathogenic fungi, which method comprises treating the seeds of the plants with one or more DMI fungicides from the groups a-d, and also soya bean seed, treated and/or coated with one or more DMI fungicides from the groups a-d.

In particular, the invention also provides the use of one or more DMI fungicides from the group consisting of:
a) triazoles:
   a.20. prothioconazole;
b) pyrimidines:
   b.1. fenarimol, b.2. nuarimol;
      and
c) pyridines:
   c.1. pyrifenox,
      for the dressing of soya bean seeds.

In particular, the invention furthermore provides a method for protecting soya bean plants against phytopathogenic fungi, which method comprises treating the seed of the plants with one or more DMI fungicides from the group consisting of
a) triazoles:
   a.20. prothioconazole;
b) pyrimidines:
   b.1. fenarimol, b.2. nuarimol;
      and
c) pyridines:
   c.1. pyrifenox.

The invention also provides soya bean seed, treated and/or coated with one or more DMI fungicides from the group consisting of
a) triazoles:
   a.20. prothioconazole;
b) pyrimidines:
   b.1. fenarimol, b.2. nuarimol;
      and
c) pyridines:
   c.1. pyrifenox.

Surprisingly, the dressing according to the invention is particularly effective against soya bean rust, in spite of the fact that this particularly aggressive plant disease is not soil-borne but wind-borne.

Moreover, using the dressing according to the invention it is possible to control fungal diseases in soya beans in an economically and ecologically advantageous manner, and also to improve plant health and vitality.

The DMI fungicides of the group a-e are known and commercially available. Details concerning the individual substances and their purchase can be found in “The Pesticide Manual”, 13th edition.

Preference is given to the DMI fungicides of group a and to prochloraz of group e.

Particular preference is given to fluquinconazole, flutriafol, ipconazole, prothioconazole, tebuconazole, triticonazole, metconazole, cyproconazole, bitertanol, triadimefon and triadimenol.

Especially preferred are fluquinconazole, flutriafol, ipconazole, prothioconazole and triticonazole.

Furthermore, it is preferred to use mixtures of two or more, in particular one, of the DMI fungicides mentioned.

Particular preference is given to the mixtures of the compounds fluquinconazole and prothioconazole.

In the above-mentioned mixtures of two active compounds, the weight ratio of the DMI fungicides in question is generally 1:0.01-100, preferably 1:0.2-20, particularly preferably 1:0.1-10.

The DMI fungicides used according to the invention, as such or in their formulations, can also be used as a mixture with other known fungicides, bactericides, acaricides, nematicides or insecticides, for example in order to broaden the activity spectrum or to prevent the development of resistances in this way. In many cases, synergistic effects result, i.e. the effectiveness of the mixture exceeds the effectiveness of the individual components.

Compounds which are suitable as mixing partners are, for example, the following:

Fungicides:

2-Phenylphenol; 8-hydroxyquinoline sulphate; acibenzolar-5-methyl; actinovate; aldimorph; amidoflumet; ampropylfos; ampropylfos-potassium; andoprim; anilazine; azoxystrobin; benalaxyl; benodanil; benomyl; benthiavalicarb-isopropyl; benzamacril; benzamacril-isobutyl; bilanafos; binapacryl; biphenyl; blasticidin-S; boscalid; bupirimate; buthiobate; butylamine; calcium polysulphide; capsimycin; captafol; captan; carbendazim; carboxin; carpropamid; carvone; chinomethionat; chlobenthiazone; chlorfenazole; chloroneb; chlorothalonil; chlozolinate; cis-1-(4-chlorophenyl)-2-(1H-1,2,4-triazol-1-yl)-cycloheptanol; clozylacon; cyazofamid; cyflufenamid; cymoxanil; cyprodinil; cyprofuram; Dagger G; debacarb; dichlofluanid; dichlone; dichlorophen; diclocymet; diclomezine; dicloran; diethofencarb; diflumetorim; dimethirimol; dimethomorph; dimoxystrobin; dinocap; diphenylamine; dipyrithione; ditalimfos; dithianon; dodine; drazoxolon; edifenphos; ethaboxam; ethirimol; etridiazole; famoxadone; fenamidone; fenapanil; fenfuram; fenhexamid; fenitropan; fenoxanil; fenpiclonil; fenpropidin; fenpropimorph; ferbam; fluazinam; flubenzimine; fludioxonil; flumetover; flumorph; fluoromide; fluoxastrobin; flurprimidol; flusulfamide; flutolanil; folpet; fosetyl-A1; fosetyl-sodium; fuberidazole; furalaxyl; furametpyr; furcarbanil; furmecyclox; guazatine; hexachlorobenzene; hymexazol; iminoctadine triacetate; iminoctadine tris(albesilate); iodocarb; iprobenfos; iprodione; iprovalicarb; irumamycin; isoprothiolane; isovaledione; kasugamycin; kresoxim-methyl; mancozeb; maneb; meferimzone; mepanipyrim; mepronil; metalaxyl; metalaxyl-M; methasulfocarb; methfiroxam; methyl 1-(2,3-dihydro-2,2-dimethyl-1H-inden-1-yl)-1H-imidazole-5-carboxylate; methyl 2-[[[cyclopropyl[(4-methoxyphenyl)imino]methyl]thio]-methyl]-α-(methoxymethylene)benzeneacetate; methyl 2-[2-[3-(4-chlorophenyl)-1-methyl-allylideneaminooxymethyl]phenyl]-3-methoxyacrylate; metiram; metominostrobin; metrafenone; metsulfovax; mildiomycin; monopotassium carbonate; myclozolin; N-(3-ethyl-3,5,5-trimethylcyclohexyl)-3-formylamino-2-hydroxybenzamide; N-(6-methoxy-3-pyridinyl)cyclopropanecarboxamide; N-butyl-8-(1,1-dimethylethyl)-1-oxaspiro[4.5]decan-3-amine; natamycin; nitrothal-isopropyl; noviflumuron; ofurace; orysastrobin; oxadixyl; oxolinic acid; oxycarboxin; oxyfenthiin; pencycuron; penthiopyrad; phosdiphen; phthalide; picobenzamid; picoxystrobin; piperalin; polyoxins; polyoxorim; procymidone; propamocarb; propanosine-sodium; propineb; proquinazid; pyraclostrobin; pyrazophos; pyrimethanil; pyroquilon; pyroxyfur; pyrrolnitrine, quinconazole; quinoxyfen; quintozene; silthiofam; sodium tetrathiocarbonate; spiroxamine; sulphur; tecloftalam; tecnazene; tetcyclacis; thicyofen; thifluzamide; thiophanate-methyl; thiram; tiadinil; tioxymid; tolclofos-methyl; tolylfluanid; triazbutil; triazoxide; tricyclamide; tricyclazole; tridemorph; trifloxystrobin; validamycin A; vinclozolin; zineb; ziram; zoxamide; (2S)- N-[2-[4-[[3-(4-chlorophenyl)-2-propynyl]oxy]-3-methoxyphenyl]ethyl]-3-methyl-2-[(methylsulphonyl)amino]butanamide; 1-(1-naphthalenyl)-1H-pyrrole-2,5-dione; 2,3,5,6-tetrachloro-4-(methylsulphonyl)pyridine; 2,4-dihydro-5-methoxy-2-methyl-4-[[[[1-[3-(trifluoromethyl)phenyl]-ethylidene]amino]oxy]methyl]phenyl]-3H-1,2,3-triazol-3-one; 2-amino-4-methyl-N-phenyl-5-thiazolecarboxamide; 2-chloro-N-(2,3-dihydro-1,1,3-trimethyl-1H-inden-4-yl)-3-pyridinecarboxamide; 3,4,5-trichloro-2,6-pyridinedicarbonitrile; 3-[(3-bromo-6-fluoro-2-methyl-1H-indol-1-yl)sulphonyl]-N,N-dimethyl-1H-1,2,4-triazole-1-sulphonamide;

and copper salts and preparations, such as Bordeaux mixture; copper hydroxide; copper naphthenate; copper oxychloride; copper sulphate; cufraneb; cuprous oxide; mancopper; oxine-copper:

Bactericides:

Bronopol, dichlorophen, nitrapyrin, nickel dimethyldithiocarbamate, kasugamycin, octhilinon, furancarboxylic acid, oxytetracyclin, probenazole, streptomycin, tecloftalam, copper sulphate and other copper preparations.

Insecticides/Acaricides/Nematicides:

Acetylcholine Esterase (AChE) Inhibitors

1.1 Carbamates,
   for example alanycarb, aldicarb, aldoxycarb, allyxycarb, aminocarb, bendiocarb, benfuracarb, bufencarb, butacarb, butocarboxim, butoxycarboxim, carbaryl, carbofuran, carbosulfan, cloethocarb, dimetilan, ethiofencarb, fenobucarb, fenothiocarb, formetanate, furathiocarb, isoprocarb, metam-sodium, methiocarb, methomyl, metolcarb, oxamyl, pirimicarb, promecarb, propoxur, thiodicarb, thiofanox, trimethacarb, XMC, xylylcarb, triazamate
1.2 Organophosphates,
   for example acephate, azamethiphos, azinphos (-methyl, -ethyl), bromophos-ethyl, bromfenvinfos (-methyl), butathiofos, cadusafos, carbophenothion, chlorethoxyfos, chlorfenvinphos, chlormephos, chlorpyrifos (-methyl/-ethyl), coumaphos, cyanofenphos, cyanophos, chlorfenvinphos, demeton-5-methyl, demeton-5-methylsulphon, dialifos, diazinon, dichlofenthion, dichlorvos/DDVP, dicrotophos, dimethoate, dimethylvinphos, dioxabenzofos, disulfoton, EPN, ethion, ethoprophos, etrimfos, famphur, fenamiphos, fenitrothion, fensulfothion, fenthion, flupyrazofos, fonofos, formothion, fosmethilan, fosthiazate, heptenophos, iodofenphos, iprobenfos, isazofos, isofenphos, isopropyl O-salicylate, isoxathion, malathion, mecarbam, methacrifos, methamidophos, methidathion, mevinphos, monocrotophos, naled, omethoate, oxydemeton-methyl, parathion (-methyl/-ethyl), phenthoate, phorate, phosalone, phosmet, phosphamidon, phosphocarb, phoxim, pirimiphos (-methyl/-ethyl), profenofos, propaphos, propetamphos, prothiofos, prothoate, pyraclofos, pyridaphenthion, pyridathion, quinalphos, sebufos, sulfotep, sulprofos, tebupirimfos, temephos, terbufos, tetrachlorvinphos, thiometon, triazophos, triclorfon, vamidothion
      Sodium Channel Modulators/Voltage-Dependent Sodium Channel Blockers
2.1 Pyrethroids,
   for example acrinathrin, allethrin (d-cis-trans, d-trans), beta-cyfluthrin, bifenthrin, bioallethrin, bioallethrin-S cyclopentyl isomer, bioethanomethrin, biopermethrin, bioresmethrin, chlovaporthrin, cis-cypermethrin, cis-resmethrin, cis-permethrin, clocythrin, cycloprothrin, cyfluthrin, cyhalothrin, cypermethrin (alpha-, beta-, theta-, zeta-), cyphenothrin, deltamethrin, empenthrin (IR isomer), esfenvalerate, etofenprox, fenfluthrin, fenpropathrin, fenpyrithrin, fenvalerate, flubrocythrinate, flucythrinate, flufenprox, flumethrin, fluvalinate, flibfenprox, gamma-cyhalothrin, imiprothrin, kadethrin, lambda-cyhalothrin, metofluthrin, permethrin (cis-, trans-), phenothrin (IR trans-isomer), prallethrin, profluthrin, protrifenbute, pyresmethrin, resmethrin, RU 15525, silafluofen, tau-fluvalinate, tefluthrin, terallethrin, tetramethrin (IR isomer), tralomethrin, transfluthrin, ZXI 8901, pyrethrins (pyrethrun)
   DDT
2.2 Oxadiazines,
   for example indoxacarb
      Acetylcholine Receptor Agonists/Antagonists
3.1 Chloronicotinyls,
   for example acetamiprid, clothianidin, dinotefuran, imidacloprid, nitenpyram, nithiazine, thiacloprid, thiamethoxam
3.2 Nicotines, bensultap, cartap
   Acetylcholine Receptor Modulators
4.1 Spinosyns,
   for example spinosad
      GABA-Controlled Chloride Channel Antagonists
5.1 Organochlorines,
   for example camphechlor, chlordane, endosulfan, gamma-HCH, HCH, heptachlor, lindane, methoxychlor
5.2 Fiproles,
   for example acetoprole, ethiprole, fipronil, pyrafluprole, pyriprole, vaniliprole
      Chloride Channel Activators
6.1 Mectins,
   for example avermectin, emamectin, emamectin-benzoate, ivermectin, milbemycin
      Juvenile Hormone Mimetics,
   for example diofenolan, epofenonane, fenoxycarb, hydroprene, kinoprene, methoprene, pyriproxifen, triprene
      Ecdyson Agonists/Disruptors
8.1 Diacylhydrazines,
   for example chromafenozide, halofenozide, methoxyfenozide, tebufenozide
      Chitin Biosynthesis Inhibitors
9.1 Benzoylureas,
   for example bistrifluoron, chlofluazuron, diflubenzuron, fluazuron, flucycloxuron, flufenoxuron, hexaflumuron, lufenuron, novaluron, noviflumuron, penfluoron, teflubenzuron, triflumuron
9.2 Buprofezin
9.3 Cyromazine
   Oxidative Phosphorylation Inhibitors, ATP Disruptors
10.1 Diafenthiuron
10.2 Organotins,
   for example azocyclotin, cyhexatin, fenbutatin-oxide
      Oxidative Phosphorylation Decouplers Acting by Interrupting the H-Proton Gradient
11.1 Pyrroles,
   for example chlorfenapyr
11.2 Dinitrophenols,
   for example binapacyrl, dinobuton, dinocap, DNOC
      Site-I Electron Transport Inhibitors
12.1 METI's,
   for example fenazaquin, fenpyroximate, pyrimidifen, pyridaben, tebufenpyrad, tolfenpyrad
12.2 Hydramethylnon
12.3 Dicofol
   Site-II Electron Transport Inhibitors
   Rotenone
      Site-III Electron Transport Inhibitors
   Acequinocyl, fluacrypyrim
      Microbial Disruptors of the Insect Gut Membrane
   Bacillus thuringiensis strains
      Fat Synthesis Inhibitors
   tetronic acids,
   for example spirodiclofen, spiromesifen
   tetramic acids,
   for example spirotetramat (CAS Reg. No.: 203313-25-1) and 3-(2,5-dimethylphenyl)-8-methoxy-2-oxo-1-azaspiro[4.5]dec-3-en-4-yl ethyl carbonate (aka: carbonic acid, 3-(2,5-dimethylphenyl)-8-methoxy-2-oxo-1-azaspiro[4.5]dec-3-en-4-yl ethyl ester, CAS Reg. No.: 382608-10-8)
   carboxamides,
   for example flonicamid
   octopaminergic agonists,
   for example amitraz
      Inhibitors of Magnesium-Stimulated ATPase,
   propargite
   benzoic acid dicarboxamides,
   for example flubendiamide
   nereistoxin analogues
   for example thiocyclam hydrogen oxalate, thiosultap-sodium
      Biologicals, Hormones or Pheromones
   azadirachtin, Bacillus spec., Beauveria spec., codlemone, Metarrhizium spec., Paecilomyces spec., thuringiensin, Verticillium spec.
      Active Compounds with Unknown or Unspecific Mechanisms of Action
23.1 Fumigants,
   for example aluminium phosphide, methyl bromide, sulphuryl fluoride
23.2 Antifeedants,
   for example cryolite, flonicamid, pymetrozine
23.3 Mite growth inhibitors,
   for example clofentezine, etoxazole, hexythiazox
23.4 Amidoflumet, benclothiaz, benzoximate, bifenazate, bromopropylate, buprofezin, quinomethionate, chlordimeform, chlorobenzilate, chloropicrin, clothiazoben, cycloprene, cyflumetofen, dicyclanil, fenoxacrim, fentrifanil, flubenzimine, flufenerim, flutenzin, gossyplure, hydramethylnone, japonilure, metoxadiazone, petroleum, piperonyl butoxide, potassium oleate, pyridalyl, sulfluramid, tetradifon, tetrasul, triarathene, verbutin

A mixture with other known active compounds, such as herbicides, or with fertilizers and growth regulators, safeners and/or semiochemicals is also possible.

Preference is given to mixtures of the above-mentioned triazoles, in particular fluquiriconazole, flutriafol, ipconazole, triticonazole and prothioconazole, with tolylfluanid or strobilurins, in particular fluoxastrobin, trifloxystrobin, azoxylstrobin and pyraclostrobin.

Particular preference is given to the mixtures fluquinconazole/tolylfluanid, fluquinconazole/fluoxastrobin, fluquinconazole/trifloxystrobin, fluquinconazole/azoxylstrobin, fluquinconazole/pyraclostrobin, prothioconazole/tolylfluanid, prothioconazole/fluoxastrobin, prothioconazole/trifloxystrobin, prothioconazole/azoxystrobin and prothioconazole/pyraclostrobin.

Preference is given to the weight ratios mentioned above.

According to the invention, the DMI fungicides are converted into the customary seed dressing formulations, such as solutions, emulsions, suspensions, powders, foams, slurries or other coating materials for seed, and also ULV formulations.

Dry treatments (preferably with addition of tackifiers, such as, for example, liquid paraffin or talc, and, if appropriate, colorants),

slurry treatments (preferably with addition of wetting agents, dispersants, emulsifiers, adhesives, inert fillers and colorants),

aqueous liquid treatments (preferably with addition of emulsifiers, dispersants, thickeners, antifreeze agents, polymers, adhesives and colorants),

solvent-based liquid treatments (with addition of solvents and colorants),

emulsion treatments (with addition of emulsifiers, solvents and colorants).

These formulations are prepared in a known manner by mixing the active compounds or active compound combinations with customary additives, such as, for example, customary extenders and also solvents or diluents, colorants, wetting agents, dispersants, emulsifiers, defoamers, preservatives, secondary thickeners, adhesives, gibberellins and water as well.

Suitable colorants that may be present in the seed dressing formulations to be used according to the invention include all colorants customary for such purposes. Use may be made both of pigments, of sparing solubility in water, and of dyes, which are soluble in water. Examples that may be mentioned include the colorants known under the designations Rhodamin B, C.I. Pigment Red 112 and C.I. Solvent Red 1.

Suitable wetting agents that may be present in the seed dressing formulations to be used according to the invention include all substances which promote wetting and are customary in the formulation of agrochemically active compounds. Preference is given to using alkylnaphthalenesulphonates, such as diisopropyl- or diisobutylnaphthalenesulphonates.

Suitable dispersants and/or emulsifiers that may be present in the seed dressing formulations to be used according to the invention include all nonionic, anionic and cationic dispersants which are customary in the formulation of agrochemically active compounds. Preference is given to using nonionic or anionic dispersants or mixtures of nonionic and anionic dispersants. Particularly suitable nonionic dispersants are ethylene oxide/propylene oxide block polymers, alkylphenol polyglycol ethers, and also tristryrylphenol polyglycol ethers and their phosphated or sulphated derivatives. Particularly suitable anionic dispersants are lignosulphonates, polyacrylic acid salts and arylsulphonate/formaldehyde condensates.

Defoamers that may be present in the seed dressing formulations to be used according to the invention include all foam-inhibiting compounds which are customary in the formulation of agrochemically active compounds. Preference is given to using silicone defoamers and magnesium stearate.

Preservatives that may be present in the seed dressing formulations to be used according to the invention include all compounds which can be used for such purposes in agrochemical compositions. By way of example, mention may be made of dichlorophen and benzyl alcohol hemiformal.

Suitable secondary thickeners that may be present in the seed dressing formulations to be used according to the invention include all compounds which can be used for such purposes in agrochemical compositions. Preference is given to cellulose derivatives, acrylic acid derivatives, xanthan, modified clays and finely divided silica.

Solvents suitable for combination with the DMI fungicides are all organic solvents which can be used in agrochemical compositions. Preference is given to ketones, such as methyl isobutyl ketone and cyclohexanone, furthermore amides, such as dimethylformamide, furthermore cyclic compounds, such as N-methylpyrrolidone, N-octylpyrrolidone, N-dodecylpyrrolidone, N-octylcaprolactam, N-dodecylcaprolactam and γ-butyrolactone, in addition strongly polar solvents, such as dimethyl sulphoxide, furthermore aromatic hydrocarbons, such as xylene, moreover esters, such as propylene glycol monomethyl ether acetate, dibutyl adipate, hexyl acetate, heptyl acetate, tri-n-butyl citrate, diethyl phthalate and di-n-butyl phthalate, and furthermore alcohols, such as ethanol, n- and i-propanol, n- and i-butanol, n- and i-amyl alcohol, benzyl alcohol and 1-methoxy-2-propanol.

Suitable polymeric additive components are customary biodegradable natural and synthetic compounds. Preference is given to polyesters, polyether esters, copolyesters, polyanhydrides, polyesterurethanes, thermoplastic polysaccharides or polysaccharide derivatives, and also polyesters, polyether esters and polyesteramides containing aliphatic and aromatic ester groupings.

Suitable adhesives that may be present in the seed dressing formulations to be used according to the invention include all customary binders which can be used in seed dressings. Polyvinylpyrrolidone, polyvinyl acetate, polyvinyl alcohol and tylose may be mentioned as being preferred.

Suitable gibberellins that may be present in the seed dressing formulations to be used according to the invention preferably include compounds of the formula 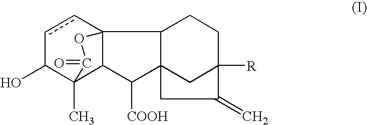
in which
R represents a hydroxyl group and
the dashed line indicates that at the position of the ring either a C—C single bond or a C═C double bond is present.

Examples that may be mentioned of gibberellins of the formula (I) include the following:

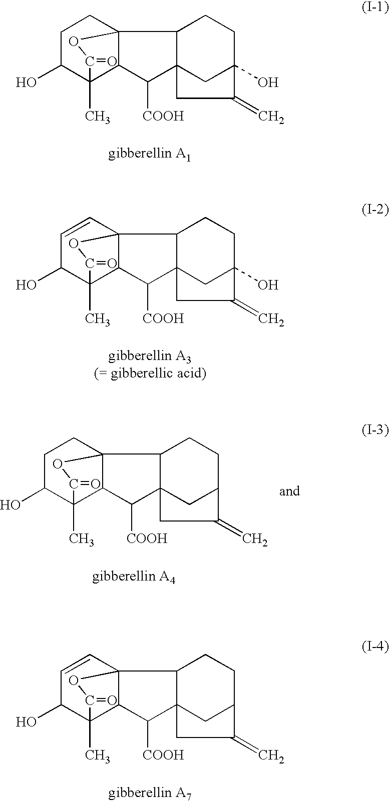

Particular preference is given to gibberellic acid of formula (I-2).

The gibberellins of the formula (I) are known (cf. R. Wegler “Chemie der Pflanzenschut-zund Schädlingsbekämpfungsmittel”, volume 2, Springer Verlag, Berlin-Heidelberg-New York, 1970, pages 401-412).

The seed dressing formulations to be used according to the invention are used either directly or after prior dilution with water for the treatment of soya bean seed. The seed dressing formulations to be used according to the invention or dilute preparations thereof can also be used for dressing seed of transgenic soya bean plants. In this context, additional synergistic effects may also arise in interaction with the substances formed by expression.

Suitable mixing equipment for treating soya bean seed with the seed dressing formulations to be used according to the invention or the preparations prepared therefrom by addition of water includes all mixing equipment which can commonly be used for dressing. The specific procedure adopted when dressing comprises introducing the seed into a mixer, adding the particular desired amount of seed dressing formulations, either as such or after prior dilution with water, and carrying out mixing until the formulation is uniformly distributed on the seed. If appropriate, this is followed by a drying operation.

The application rate of the seed dressing formulations to be used according to the invention may be varied within a relatively wide range. It depends on the respective content of the active compounds in the formulation and on the seed. In general, the application rates of DMI fungicides or combination thereof are between 0.001 and 50 g per kilogram of seed, preferably between 0.01 and 15 g per kilogram of seed.

In addition to the control of Phakopsora,in particular Phakopsora pachyrhizi and Phakopsora meibomiae, the seed dressings used according to the invention can be employed, for example, for controlling Plasmodiophoromycetes, Oomycetes, Chytridiomycetes, Zygomycetes, Ascomycetes, Basidiomycetes, Deuteromycetes, Pseudomonadaceae, Rhizobiaceae, Enterobacteriaceae, Corynebacteriaceae and Streptomycetaceae.

Some pathogens causing fungal and bacterial diseases which come under the generic names listed above may be mentioned by way of example, but not by way of limitation:

Xanthomonas species, such as, for example, Xanthomonas campestris pv. oryzae;

Pseudomonas species, such as, for example, Pseudomonas syringae pv. lachrymans;

Erwinia species, such as, for example, Erwinia amylovora;

Phytophthora species, such as, for example, Phytophthora sojae;

Pseudoperonospora species, such as, for example, Pseudoperonospora humuli or Pseudoperonospora cubensis;

Peronospora species, such as, for example, Peronospora brassicae or Peronospora manshurica;

Erysiphe species, such as, for example, Erysiphe polygoni;

Podosphaera species, such as, for example, Podosphaera leucotricha;

Puccinia species, such as, for example, Puccinia recondita;

Sclerotinia species, such as, for example, Sclerotinia sclerotiorum;

Ustilago species, such as, for example, Ustilago nuda or Ustilago avenae;

Pellicularia species, such as, for example, Pellicularia sasakii;

Fusarium species, such as, for example, Fusarium culmorum;

Septoria species, such as, for example, Septoria nodorum or Septoria glycines;

Leptosphaeria species, such as, for example, Leptosphaeria nodorum;

Cercospora species, such as, for example, Cercospora kikuchii;

Alternaria species, such as, for example, Alternaria brassicae.

Phomopsis species, such as, for example, Phomopsis longicola; Colletotrichum species, such as, for example, Celletotrichum truncatum;

Rhizoctoinia species, such as, for example, Rhicoctoinia solani;

and in particular

Microsphera species, such as Microsphera manshurica.

The seed dressings used according to the invention are particularly suitable for controlling soya bean rust.

The dressings according to the invention also exhibit a very strong invigorating action in plants. Accordingly, they are suitable for mobilizing the internal defenses of the plant against attack by unwanted microorganisms.

In the present context, plant-invigorating (resistance-inducing) compounds are to be understood as meaning substances which are capable of stimulating the defense system of plants such that, when the treated plants are subsequently inoculated with unwanted microorganisms, they display substantial resistance to these microorganisms.

In the present case, unwanted microorganisms are to be understood as meaning phytopathogenic fungi and bacteria. The compounds according to the invention can thus be used to protect plants within a certain period of time after treatment against attack by the pathogens mentioned. The period of time for which this protection is achieved generally extends for 1 to 70 days, preferably 1 to 35 days, from sowing.

The invention is illustrated in more detail by the examples, without being limited to these.

### EXAMPLE 1

### Action of Fluquinconazole against Phakopsora Pachyrhizi

Seed Dressing: Fluquinconazole 25 WPC (Wettable Powder)

For dressing, the seed was shaken for 1 minute with the seed dressing in a closed plastic bag.

The seed was sown in a field (4 plots, plot size in each case 10 m², 40 seeds per m²) and, one day after sowing, artificially irrigated.

The natural degree of infection in the control was, on average, 75%.

The efficacy is expressed in percent. 0% means an efficacy which corresponds with that of the control, whereas an efficacy of 100% means that no infection is observed.

| Application rate | | |
|---|---|---|
| [g of fluquinconazole/ | Duration | Efficacy |
| 100 kg of seed] | [days] | [%] |
| 50 | 22 | 100 |
| 50 | 33 | 100 |
| 50 | 37 | 100 |
| 5 | 28 | 100 |

### EXAMPLE 2

### Action of Prothioconazole against Phakopsora Pachyrhizi

Seed Dressing: Prothioconazole

For dressing, the seed was shaken for 1 minute with the seed dressing in a closed plastic bag.

The seed was sown in a field (4 plots, plot size in each case 10 m², 40 seeds per m²) and, one day after sowing, artificially irrigated.

The natural degree of infection in the control was, on average, 75%.

The efficacy is expressed in percent. 0% means an efficacy which corresponds with that of the control, whereas an efficacy of 100% means that no infection is observed.

| Application rate | | |
|---|---|---|
| [g of prothioconazole/ | Duration | Efficacy |
| 100 kg of seed] | [days] | [%] |
| 5 | 28 | 100 |
| 10 | 28 | 100 |

### EXAMPLE 3

### Action of Flutriafol against Phakopsora Pachyrhizi

Seed Dressing: Flutriafol/Carbendazim/Imidacloprid

For dressing, the seed was shaken for 1 minute with the seed dressing in a closed plastic bag.

The seed was sown in a field (4 plots, plot size in each case 10 m², 40 seeds per m²) and, one day after sowing, artificially irrigated.

Soy bean plants the seeds of which had been treated with a mixture of the fungicide carbendazim (100 g/100 kg of seed) and the insecticide imidacloprid (120 g/100 kg of seed) were used as control.

The natural degree of infection in the control was, on average, 75%.

The efficacy is expressed in percent according to Abbot's formula ([(efficacy control−efficacy inventive example)/efficacy control]*100). 0% means an efficacy which corresponds with that of the control, whereas an efficacy of 100% means that no infection is observed.

| Application rate | | |
|---|---|---|
| [g flutriafol/g | | |
| carbendazim/g Imidacloprid/ | Duration | Efficacy |
| 100 kg of seed] | [days] | [%] |
| 7.5/100/120 | 19 | 99.8 |
| 7.5/100/120 | 25 | 100 |

1. A method of protecting soya beans against soya bean rust comprising applying one or more demethylation inhibitor (DMI) fungicides selected from the group consisting of:
a) triazoles:
a.1. azaconazole, a.2. bitertanol, a.3. bromuconazole, a.4. cyproconazole, a.5. difenoconazole, a.6. diniconazole, a.7. epoxiconazole, a.8. fenbuconazole, a.9. fluquinconazole, a.10. flusilazole, a.11. flutriafol, a.12. hexaconazole, a.13. imibenconazole, a.14. ipconazole, a.15. metconazole, a.16. myclobutanil, a.17. paclobutrazol, a.18. penconazole, a.19. propiconazole, a.20. prothioconazole, a.21. simeconazole, a.22. tebuconazole, a.23. tetraconazole, a.24. triadimenol, a.25. triticonazole;
b) pyrimidines:
b.1. fenarimol, b.2. nuarimol;
c) pyridines:
c.1. pyrifenox;
d) imidazoles:
d.1. imazalil, d.2. oxpoconazole fumarate, d.3. peforazoate, d.4. prochloraz, d.5. triflumizole; and mixtures thereof

2. The method according to claim 1, wherein said DMI fungicide is selected from the group consisting of fluquinconazole, flutriafol, ipconazole, prothioconazole and triticonazole.

3. A method for protecting soya bean plants against soya bean rust, comprising applying one or more DMI fungicides selected from the group consisting of:
a) triazoles:
a.1. azaconazole, a.2. bitertanol, a.3. bromuconazole, a.4. cyproconazole, a.5. difenoconazole, a.6. diniconazole, a.7. epoxiconazole, a.8. fenbuconazole, a.9. fluquinconazole, a.10. flusilazole, a.11. flutriafol, a.12. hexaconazole, a.13. imibenconazole, a.14. ipconazole, a.15. metconazole, a.16. myclobutanil, a.17. paclobutrazol, a.18. penconazole, a.19. propiconazole, a.20. prothioconazole, a.21. simeconazole, a.22. tebuconazole, a.23. tetraconazole, a.24. triadimenol, a.25. triticonazole;
b) pyrimidines:
b.1. fenarimol, b.2. nuarimol;
c) pyridines:
c.1. pyrifenox:
d) imidazoles:
d.1. imazalil, d.2. oxpoconazole fumarate, d.3. peforazoate, d.4. prochloraz, d.5. triflumizole; and mixtures thereof
to the seed of said plants.

4. The method according to claim 3 wherein said one or more DMI fungicides is selected from the group consisting of fluquinconazole, flutriafol, ipconazole, prothioconazole and triticonazole.

5. A method of protecting soya beans against soya bean rust comprising applying one or more DMI fungicides selected from the group consisting of:
a) triazoles:
a.20. prothioconazole;
b) pyrimidines:
b.1. fenarimol, b.2. nuarimol;
c) pyridines:
c.1. pyrifenox; and mixtures thereof to said soya beans.

6. The method according to claim 5 wherein said DMI fungicide is prothioconazole.

7. A method for protecting soya bean plants against phytopathogenic fungi, which method comprises applying one or more DMI fungicides selected from the group consisting of:
a) triazoles:
a.20. prothioconazole;
b) pyrimidines:
b.1. fenarimol, b.2. nuarimol;
c) pyridines:
c.1. pyrifenox; and mixtures thereof to the seed of said plants.

8. The method according to claim 7 where the DMI fungicide is prothioconazole.

9. Soya bean seed, that has been treated, coated or a combination thereof with one or more DMI fungicides selected from the group consisting of:
a) triazoles:
a.20. prothioconazole;
b) pyrimidines:
b.1. fenarimol, b.2. nuarimol,
c) pyridines:
c.1. pyrifenox; and mixtures thereof.

10. The soya bean seed according to claim 9, wherein said DMI fungicide is prothioconazole.
